(19) United States
(12) Patent Application Publication
(10) Pub. No.: US 2007/0073324 A1
(43) Pub. Date: Mar. 29, 2007

(54) Method and surgical tool for forming scleral tunnels

(76) Inventor: Georges Baikoff, Marseiue (FR)

Correspondence Address: SHELDON MAK ROSE & ANDERSON PC, 225 SOUTH LAKE AVENUE, 9TH FLOOR, PASADENA, CA, 91101, US

(21) Appl. No.: 11/239,953

(22) Filed: Sep. 29, 2005

(51) Int. Cl. A61F 9/00

(52) U.S. Cl. 606/166

(57) ABSTRACT

A method for making an incision into the sclera of an eye includes the steps of (a) applying pressure to a portion of the surface of the sclera to flatten that portion of the sclera, (b) cutting an incision in the sclera with a linear blade along a line parallel to the flattened surface of the sclera, and (c) releasing the pressure on the portion of the surface of the sclera to allow the sclera to return to its natural curvature. By this method, the incision cut into the sclera in step (b) takes on a curvature which parallels the natural curvature of the sclera. The method can be carried out using a tool which includes (a) a generally smooth scleral flattening surface disposed within a flat, flattening surface plane, and (b) a generally linear incision blade having a sharpened free end. The incision blade is disposed generally parallel with the flattening surface and spaced apart from the flattening surface by a distance of between about 0.5 mm and about 0.8 mm.

### BACKGROUND OF THE INVENTION

Presbyopia is a loss or reduction of the accompanying power of the eye which takes place when a person ages. FIG. 1 is a diagrammatic representation of an eye 1 showing the lens 2 enclosed in the lens sac 3 and suspended from the ciliary body 4 by means of the zonule 5. The ciliary body 4 lines the internal surface of the sclera 6 about a ring located on the average at a latitude distance by 2 to 3 mm from the limbus 7, measured along the optical axis.

New methods for treating presbyopia have recently been disclosed wherein implant elements are disposed within small tunnels formed within the sclera of the patient's eyes. Once disposed in the scleral tunnels, the implants act on the sclera to enhance the ability of the patient's lens to contract, thereby diminishing the presbyopia condition. Examples of such new methods are disclosed, for example, in my previously-issued patents, U.S. Pat. Nos. 6,682,560 and 6,692,524, the entireties of which are incorporated herein by this reference.

FIG. 2 illustrates one of these new methods. An implant element 8 is surgically disposed within an incision tunnel 9 formed in the sclera 6 opposite the zonule 5. The tunnel 9 is disposed at a depth of about 600 μ below the surface of the sclera 6. The tunnel 9 is typically about 8 mm long and about 3 mm wide. The implant element 8 favorably effects the adjustment of the lens shape by the sclera 6 and the zonule 7 to minimize the effects of presbyopia.

In such new methods, it is critical that the implants be disposed at precise locations within the sclera, including at a precise distance below the surface of the sclera. Accordingly, it is important that the small tunnels be disposed parallel to the sclera surface and formed at the precise distance below the surface of the sclera. Since the sclera is curved, it is therefore important to form the small tunnels along a curved line which parallels the curvature of the sclera. By forming the tunnels along such curved lines, it can be assured that the small tunnels are uniformly formed at the precise distance below the surface of the sclera.

One could attempt to form the small tunnels along the appropriate curved lines using surgical blades having the precise curvature of the desired curved line. Unfortunately, the curvature of all scleras differ from one another, so that forming the small tunnels with a curved surgical blade would necessitate the manufacture of a large set of surgical blades, each one differing slightly in curvature. Moreover, choosing a cutting blade having a curvature which coincides with the curvature of any particular sclera would require precise measurement methods and tools for measuring the curvature of each sclera. Accordingly, the forming of the small tunnels along the appropriate curved line within the sclera using curved surgical blades is unduly complicated and expensive.

Therefore, there is a need for a surgical method and tools for forming the small tunnels along a path parallel with the curvature of the sclera while avoiding the problems associated with the use of curved surgical blades discussed above.

### SUMMARY OF THE INVENTION

The invention satisfies this need. The invention is a method for making an incision into the sclera of an eye whereby the incision has a curvature which parallels the natural curvature of the sclera, the method comprising the steps of (a) applying pressure to a portion of the surface of the sclera to flatten that portion of the sclera, (b) cutting an incision in the sclera with a linear blade along a line parallel to the flattened surface of the sclera, and (c) releasing the pressure on the portion of the surface of the sclera to allow the sclera to return to its natural curvature, whereby the incision cut into the sclera in step (b) takes on a curvature which parallels the natural curvature of the sclera.

The invention is also a tool for accomplishing the method of the invention. The tool comprises (a) a generally smooth scleral flattening surface disposed within a flat, flattening surface plane, and (b) a generally linear incision blade having a sharpened free end, the incision blade being disposed generally parallel with the flattening surface and spaced apart from the flattening surface by a distance of between about 0.5 mm and about 0.8 mm.

### DRAWINGS

These and other features, aspects and advantages of the present invention will become better understood with reference to the following description, appended claims and accompanying drawings where:

FIG. 1 is a schematic view in cross-section of an eye;

FIG. 2 is a fragmentary cross-sectional view of an eye in which is implanted a corrective element for the treatment of presbyopia;

FIG. 3 is a partial plan view of a surgical tool having features of the invention;

FIG. 4 is a partial side view of the instrument illustrated in FIG. 3;

FIGS. 5A-5B illustrate, in diagrammatic form, the use of a surgical tool having features of the invention to create an incision within the sclera of an eye;

FIGS. 5E-5H illustrate, in diagrammatic form, the use of an alternative surgical tool having features of the invention to create an incision within the sclera of an eye;

FIGS. 6A-6C illustrate different embodiments of the surgical tool illustrated in FIGS. 5E-5H, each embodiment having a different handle configuration;

FIG. 7A is a cross-sectional side view of another embodiment of a surgical tool having features of the invention, showing the blade of the tool in a retracted position;

FIG. 7B is a cross-sectional side view of the surgical tool embodiment illustrated in FIG. 7A, showing the blade of the tool in an extended position;

FIG. 7C is a cross-sectional side view of the use of the surgical tool illustrated in FIG. 7A to make an incision in the sclera of an eye;

FIG. 8 is a cross-sectional side view of yet another embodiment of a surgical tool having features of the invention; and

FIG. 9 is a cross-sectional side view illustrating the use of the surgical tool illustrated in FIG. 8 to make an incision within the sclera of an eye.

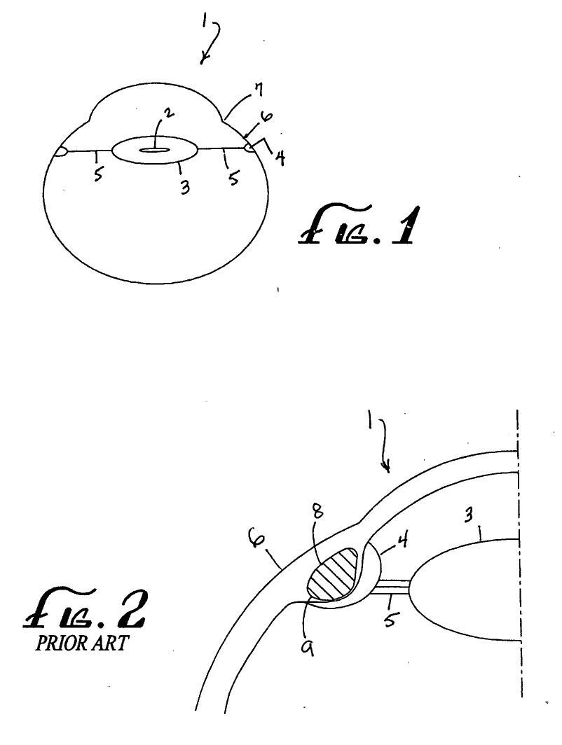

### DETAILED DESCRIPTION

The following discussion describes in detail one embodiment of the invention and several variations of that embodiment. This discussion should not be construed, however, as limiting the invention to those particular embodiments. Practitioners skilled in the art will recognize numerous other embodiments as well.

The invention is a method for making an incision 9 into the sclera 6 of an eye 1 such that the incision 9 has a curvature which closely parallels the natural curvature of the sclera 6. The invention comprises the steps of (a) applying pressure to a portion of the surface 10 of the sclera 6 to flatten that portion of the sclera 10, (b) cutting an incision 9 in the sclera 6 with a linear incision blade 12 along a line parallel to the flattened surface 10 of the sclera 6, and (c) releasing the pressure on the flattened portion of the surface 10 of the sclera 6 to allow the sclera 6 to return to its natural curvature. In the invention, the releasing of pressure on the flattened surface of the sclera 6 in step (c) causes the incision 9 in the sclera 6 made in step (b) to take on a curvature which parallels the natural curvature of the sclera 6.

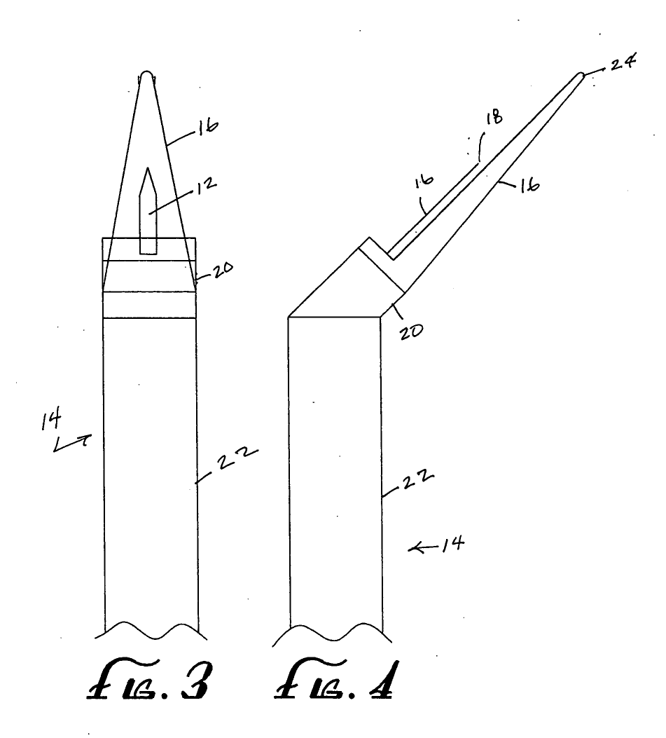

The invention can be accomplished using a tool 14 such as the tools 14 illustrated in FIGS. 3-9. In all embodiments, the tool 14 comprises (a) a generally smooth scleral flattening surface 16 disposed within a flat, flattening surface plane, and (b) a rigid, generally linear incision blade 12, the incision blade 12 being disposed generally parallel with the scleral flattening surface 16 and spaced apart from the scleral flattening surface 16 by a distance of between about 0.5 mm and about 0.8 mm.

FIGS. 3 and 4 illustrate a first embodiment of such a tool 14. The tool 14 illustrated in FIGS. 3 and 4 has an incision blade 12 with a width between about 1.1 mm and about 1.4 mm and a thickness of about 100 microns. The incision blade 12 has a sharp incision blade tip 18. The sides of the incision blade 12, however, are preferably blunt, so that the incision tunnel 9 is not opened too widely on the sides. Typically, the incision blade 12 is made of steel, sapphire or diamond.

The incision blade 12 is disposed within a retaining element 20 which is attached to, or which doubles as, a handle 22. The incision blade tip 18 extends beyond the retaining element 20 by a distance of at least about 8 mm.

The tool 14 illustrated in FIGS. 3 and 4 also comprises a scleral flattening surface 16 which attached to, or formed integrally with, the retaining element 20. The scleral flattening surface 16 has a scleral flattening surface tip 24. Those of ordinary skill in the art will recognize that other shapes for the scleral flattening surface tip 24 can also be used.

The scleral flattening surface 16 can be transparent and have graduations, in order to be able to confirm that sufficient pressure is applied to the scleral surface 10 before the incision 9 is made. Graduations can also be provided in the scleral flattening surface 16 to indicate the distance between the tunnel 9 and the limbus 7.

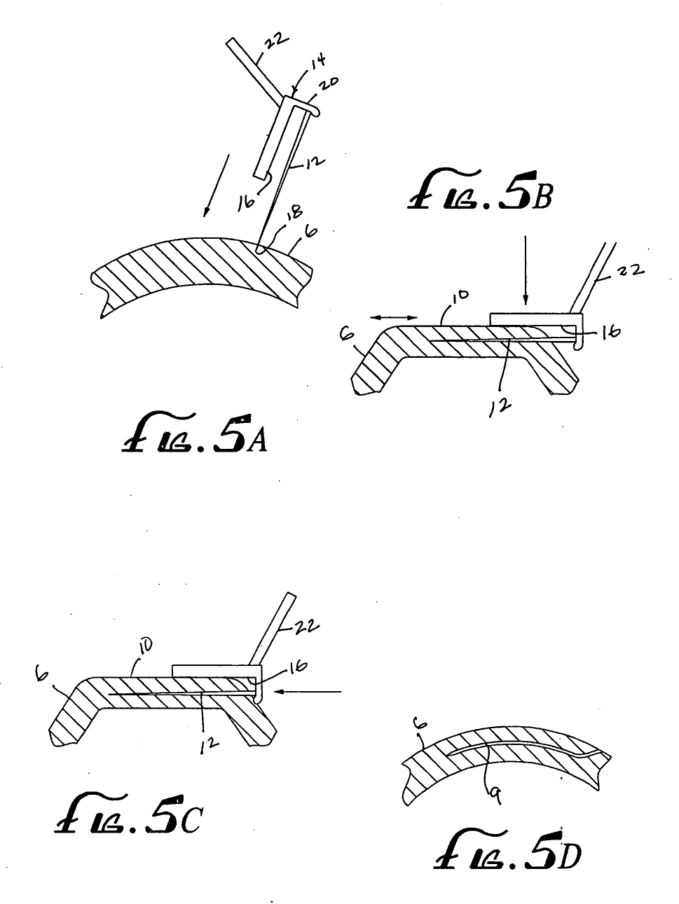

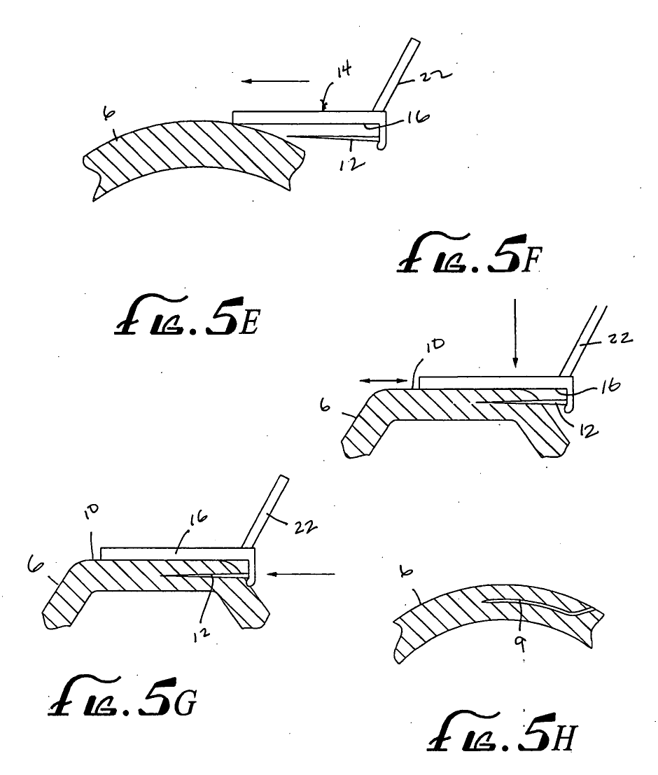

In the embodiment illustrated in FIGS. 3 and 4, the scleral flattening surface tip 24 extends outwardly from the retaining element 20 a distance of between about 1 mm and about 2 mm beyond the incision blade tip 18. The reason for this is illustrated in FIGS. 5E-5H, described below.

Typically, the handle 22 of the tool 14, the retaining element 20 and the scleral flattening surface 16 are made of steel to facilitate the resterilization of the tool. Those of ordinary skill in the art will recognize that other materials can be used, as well.

FIGS. 5A-5B illustrate diagrammatically a second embodiment of the tool 14 and its use in making an incision into the sclera 6 of the eye 1. The embodiment of the tool 14 illustrated in FIGS. 5A-5B comprises a handle 22, a retaining element 20, an incision blade 12 and a scleral flattening surface 16. Unlike the embodiment of the tool 14 illustrated in FIGS. 3 and 4, in the embodiment of the tool 14 illustrated in FIGS. 5A-5B, the incision blade tip 18 extends outwardly a distance of between about 0.5 mm and about 2 mm, and typically between about 1 mm and about 2 mm, beyond the scleral flattening surface tip 24.

The embodiment of the tool 14 illustrated in FIGS. 5A-5B can be used to make an incision 9 into the sclera 6 of the eye 1 by the following steps. First, the incision blade 12 is disposed approximately orthogonally to the surface of the sclera 6 as illustrated in FIG. 5A. While the incision blade 12 is disposed orthogonally to the surface of the sclera 6, the incision blade tip 18 is contacted with the surface of the sclera 6 and the incision blade tip 18 is used to make an initial portion of the incision 9 in the sclera 6. Next, the scleral flattening surface 16 is used to apply pressure to a portion of the surface 10 of the sclera 6 to flatten that portion of the scleral surface 10, and the incision blade 12 is rotated so that the incision blade 12 is disposed parallel to the flattened surface portion 10 of the sclera 6. Next, the incision blade 12 is moved parallel to the flattened portion 10 of the sclera 6 to continue the insertion of the insertion blade 12 into the scleral 6 to finish making the incision 9 in the sclera 6 (as illustrated in FIGS. 5B and 5C). Lastly, the incision blade 12 is retracted from the sclera 6 and the sclera flattening surface 16 is removed from contact with the surface of the sclera 6, leaving behind a tunnel 9 into which an implant 8 can be disposed in the treatment of presbyopia. When this is done, the surface 10 of the sclera 6 returns to its natural arcuate shape. When the sclera 6 returns to its arcuate shape, the tunnel 9 formed within the sclera 6 is also arcuate and is generally parallel to the curvature to the surface of the sclera 6 (as illustrated in FIG. 5D).

FIGS. 5E-5H illustrate another embodiment of the tool 14 of the invention and its use. The embodiment illustrated in FIGS. 5E-5H is similar to the design of the embodiment illustrated in FIGS. 3 and 4, in that the scleral flattening surface tip 24 extends outwardly a distance greater than the incision blade tip 18. The use of the tool 14 illustrated in FIGS. 5E-5H is described as follows: As illustrated in FIG. 5E, the scleral flattening surface 16 is first used to press down on a portion of the surface 10 of the sclera 6 to flatten it. Next, the tool 14 is moved laterally, parallel with the flattened surface 10 of the sclera 6, so that the incision blade 12 forms a tunnel 9 which is generally parallel to the flattened surface 10 of the sclera 6 (as illustrated in FIGS. 5F and 5G). Once the tunnel 9 is formed within the sclera 6, the incision blade 12 is retracted from the sclera 6 and the sclera flattening surface 16 is removed from contact with the surface of the sclera 6. When this is accomplished, the surface 16 of the sclera 6 returns to its natural arcuate shape as illustrated in FIG. 5H. As also illustrated in FIG. 5H, the tunnel 9 formed in the sclera 6 takes on a curvature which generally parallels the natural curvature of the surface 16 of the sclera.

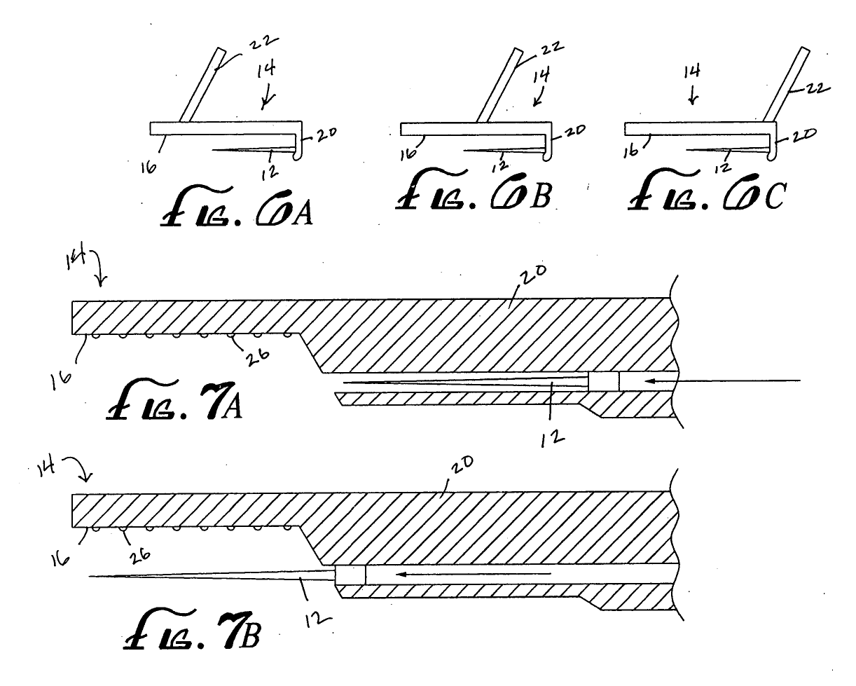

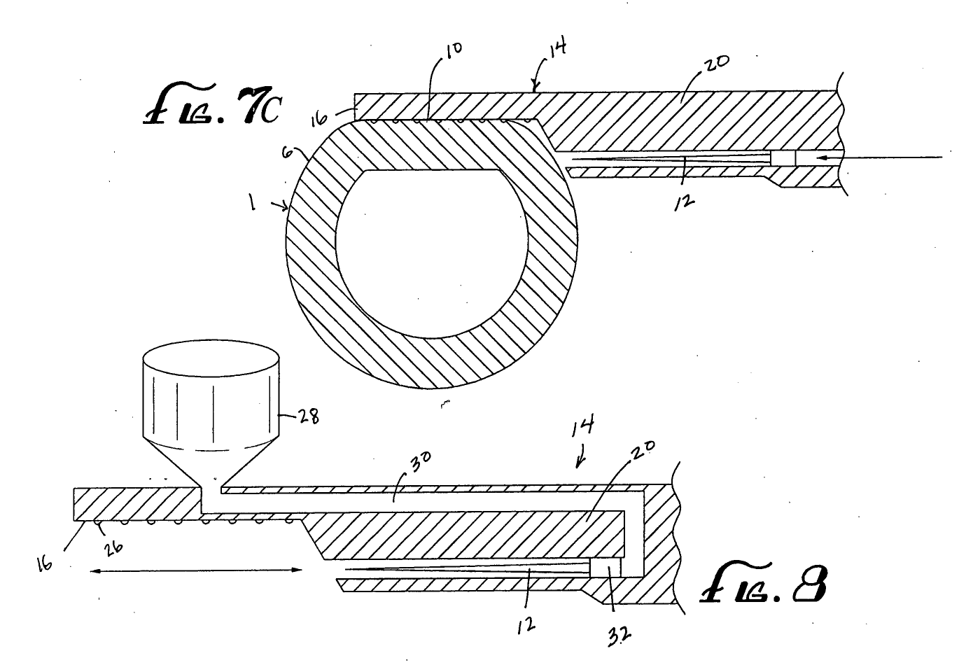

FIGS. 6A-6C illustrate three different additional embodiments of the tool 14 of the invention. Each of these three embodiments differs from one another by the placement of the handle 22 on the tool 14.

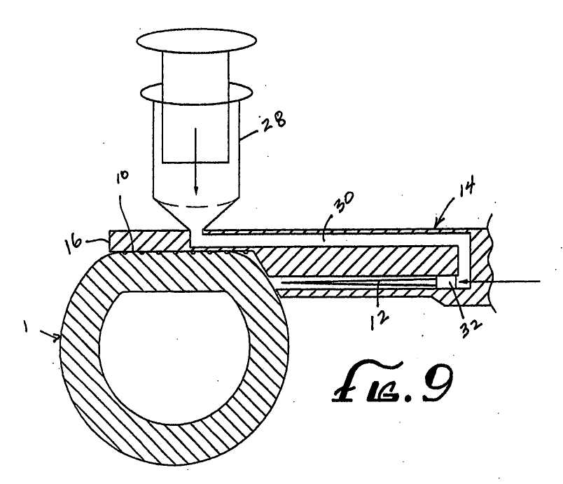

FIGS. 7A and 7B illustrate yet another embodiment of the tool 14 of the invention. In this embodiment, the incision blade 12 is slidably retained within the retaining element 20 and is capable of alternatively moving between an extended position (illustrated in FIG. 7B) and a retracted position (illustrated in FIG. 7A). In this embodiment, it is preferred that the scleral flattening surface 16, although being generally smooth, defines tiny traction elements 26 for minimizing slippage of the scleral flattening surface 16 along the surface 16 of the sclera 6. The length of the scleral flattening surface 16 in this embodiment is typically about 10 mm.

FIG. 7C illustrates the use of the embodiment shown in FIGS. 7A and 7B to create an incision 9 within the sclera 6. The scleral flattening surface 6 is placed in contact with the surface of the sclera 6 and pressed down to flatten that portion of the scleral surface 10. Next, the incision blade 12 is caused to move from its retracted position to its extended position. As this is being accomplished, the incision blade 12 forms an incision 9 within the sclera 6 which is generally parallel to the flattened portion of the scleral surface 10. Thereafter, when the incision blade 12 is moved to the retracted position, and the scleral flattening surface 16 is disengaged from the surface of the sclera 6, the surface 16 of the sclera 6 returns to its natural curvature. The incision 9 within the sclera 6 then become curved and will be generally parallel to the curvature of the surface 16 of the sclera 6.

FIG. 8 illustrates yet another embodiment of the tool 14 of the invention. In this embodiment, a pump 28, such as a syringe, is used in a version of the embodiment illustrated in FIGS. 7A and 7B. The pump 28 is in fluid communication via an internal passageway 30 in the tool 14 to a piston 32 attached to the rearmost end of the incision blade 12. When the pump 28 and the passageway 30 are filled with a liquid, the pump 28 can apply hydraulic pressure to the piston 32 at the rear of the incision blade 12. By application of hydraulic pressure to the piston 32, the incision blade 12 can be caused to move from its retracted position to its extended position (as illustrated in FIG. 9).

Having thus described the invention, it should be apparent that numerous structural modifications and adaptations may be resorted to without departing from the scope and fair meaning of the instant invention as set forth hereinabove and as described hereinbelow by the claims.

What is claimed is:

1. A method for making an incision into the sclera of an eye whereby the incision has a curvature which parallels the natural curvature of the sclera, the method comprising the steps of:
(a) applying pressure to a portion of the surface of the sclera to flatten that portion of the sclera;
(b) cutting an incision in the sclera with a linear blade along a line parallel to the flattened surface of the sclera; and
(c) releasing the pressure on the portion of the surface of the sclera to allow the sclera to return to its natural curvature;
whereby the incision cut into the sclera in step (b) takes on a curvature which parallels the natural curvature of the sclera.

2. The method of claim 1 wherein the applying of pressure to a portion of the surface of the sclera in step (a) and the cutting of an incision in the sclera with a linear blade in step (b) are both accomplished using a tool which comprises:
(i) a generally smooth scleral flattening surface disposed within a flat, flattening surface plane, and
(ii) a generally linear incision blade having a sharpened free end, the incision blade being disposed generally parallel with the flattening surface and spaced apart from the flattening surface by a distance of between about 0.5 mm and about 0.8 mm.

3. The method of claim 2 wherein the scleral flattening surface has a scleral flattening surface tip, wherein the incision blade has an incision blade tip and wherein the scleral flattening surface tip extends outwardly beyond the incision blade tip by a distance between about 0.5 mm and about 2 mm.

4. The method of claim 2 wherein the scleral flattening surface has a scleral flattening surface tip, wherein the incision blade has an incision blade tip and wherein the incision blade tip extends outwardly beyond the scleral flattening surface tip by a distance between about 1 mm and about 2 mm.

5. The method of claim 4 wherein the cutting of an incision in the sclera is accomplished by the steps of (i) disposing the incision blade approximately orthogonally to the surface of the sclera, (ii) contacting the surface of the sclera with the incision blade tip to make the initial portion of the incision in the sclera, (iii) using the scleral flattening surface to apply pressure to a portion of the surface of the sclera to flatten that portion of the sclera and rotating the incision blade so that the incision blade is disposed approximately parallel to the flattened surface portion of the sclera, and (iv) continuing to insert the incision blade into the sclera to finish making the incision in the sclera.

6. The method of claim 2 further comprising an incision blade retaining element and wherein the incision blade is slidably retained within the incision blade retaining element and alternatively moved between an extended position and a retracted position.

7. The method of claim 2 wherein the incision blade is movable between the retracted position and the extended position by hydraulic pressure.

8. A tool for making an incision for the insertion of a corrective element into the sclera of an eye, the tool comprising:
(a) a generally smooth scleral flattening surface disposed within a flat, flattening surface plane; and
(b) a generally linear incision blade having a sharpened free end, the incision blade being disposed generally parallel with the flattening surface and spaced apart from the flattening surface by a distance of between about 0.5 mm and about 0.8 mm.

9. The tool of claim 8 wherein the scleral flattening surface has a scleral flattening surface tip, wherein the incision blade has an incision blade tip and wherein the scleral flattening surface tip extends outwardly beyond the incision blade tip by a distance between about 0.5 mm and about 2 mm.

10. The tool of claim 8 wherein the scleral flattening surface has a scleral flattening surface tip, wherein the incision blade has an incision blade tip and wherein the incision blade tip extends outwardly beyond the scleral flattening surface tip by a distance between about 1 mm and about 2 mm.

11. The tool of claim 8 further comprising an incision blade retaining element and wherein the incision blade is slidably retained within the incision blade retaining element and alternatively moved between an extended position and a retracted position.

12. The tool of claim 11 wherein the incision blade is movable between the retracted position and the extended position by hydraulic pressure.
